(19) United States
(12) Patent Application Publication
(10) Pub. No.: US 2004/0133095 A1
(43) Pub. Date: Jul. 8, 2004

(54) Methods and devices for detecting abnormal tissue cells

(76) Inventors: Robert J. Dunki-Jacobs, Mason, OH (US); Yoav Avidor, Cincinnati, OH (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/713,407

(22) Filed: Nov. 14, 2003

Related U.S. Application Data
(60) Provisional application No. 60/426,211, filed on Nov. 14, 2002.

(51) Int. Cl. A61B 5/00

(52) U.S. Cl. 600/407; 600/431; 600/436

(57) ABSTRACT

Devices and methods are provided for identifying tissue cells, such as cancerous cells. The device can include a swallowable capsule having a detector. A patient can be given a substance which includes a marker material (such as a radioactive marker or a magnetic marker material), and which substance can be preferentially bound to or otherwise associated with the particular cell type.

### FIELD OF THE INVENTION

The present invention relates to medical devices and methods, and more particularly to devices and methods for detecting abnormal tissue cells, such as cancerous tissue cells.

### BACKGROUND OF THE INVENTION

Colorectal cancer is the third most common cancer in the United States, and the second in terms of annual cancer mortality. Each year, over 130,000 Americans are diagnosed with this disease. Fortunately, unlike many other cancers the prognosis associated with a diagnosis of colorectal cancer can be optimistic if the cancer is discovered early. Indeed, when discovered at an early stage, the 5-year survival and cure are over 90%. However, when the cancer is uncovered at a more advanced stage prognosis is dismal. Hence the medical community's belief in the clinical and economic value of general screening for colorectal cancer, which is recommended (and reimbursed accordingly) in the United States for every adult over 50 years-of age.

Yet despite its proven value, the general population, due to several issues that will be highlighted herewith has not adopted colorectal cancer screening. These impediments to mass screening reduce its penetration considerably. Thus, the overall survival of colorectal cancer patients is only 40%, a situation that can be much improved upon if a better screening modality emerges.

Current screening modalities for colorectal cancer include occult fecal blood (Hemoccult), barium enema, sigmoidoscopy, colonoscopy, and experimental technologies such as CT Virtual Colonography and fecal DNA testing. These modalities can detect some small and early cancers. However, like any diagnostic modality, their adoption as a mass screening tool depends on their ability to provide benefits such as low cost testing, reliable sensitivity in detecting malignancy, and good specificity as to indicating the location of the malignancy in the patient's body.

Fecal occult blood screening can be easy to administer and relatively low cost, but also associated with low sensitivity for cancer, between 5-35% depending on the size and stage of the tumor. Additionally, patients find repeated retrieval of specimens from fresh stool objectionable and demeaning.

Sigmoidoscopy can provide higher sensitivity for disease in the left (descending) colon. Only 40-50% of potentially malignant lesions are detectable by a sigmoidoscope. Accuracy of sigmoidoscopy has been shown to be sensitive to physician expertise. Additionally, patients find the total colon cleansing regimen (“bowel prep”) and pre-procedure dietary restrictions objectionable, uncomfortable and inconvenient.

Colonoscopy provides relatively high sensitivity and specificity. However, colonoscopy requires advanced physician expertise that increases costs and limits its use in a mass-scale setting. The additional cost and risks associated with the administration of conscious sedation also limit adoption of this procedure as a screening methodology. As with sigmoidoscopy, patients find the total colon cleansing regimen (“bowel prep”) and pre-procedure dietary restrictions objectionable, uncomfortable and inconvenient.

Virtual colonoscopy based on 3D Computed Tomography or Magnetic Resonance image sets is currently under development. While the sensitivity and specificity of this approach is still being debated, either imaging modality would require a bowel prep and colon insuflation (an uncomfortable part of the sigmoidoscopy and colonoscopy procedure) in order to achieve acceptable results.

Fecal DNA testing promises more sensitivity than fecal occult blood testing. These results have not been proven to date. Regardless, the specimen collection mechanism is substantially the same as that for fecal occult blood and therefore patients will find retrieval of specimens from fresh stool objectionable and demeaning.

### SUMMARY OF THE INVENTION

In one embodiment, the present invention comprises a method for detecting target cell types in a patient, such as in a procedure for diagnosis or screening for colon cancer. The method can include the steps of marking target cells with a signal emitting substance while leaving surrounding non-target cells substantially free of the signal emitting substance; and introducing a detector into a naturally occurring body lumen, such as the gastrointenstinal tract in the patient to determine to the location of the target cells.

A method according to the present invention can include administering to a patient, such as by injection, a material comprising at least one signal emitting substance and at least one substance having an affinity for a target cell type; providing a detector capable of detecting signals emitted by the substance; and introducing a detector enclosed in a swallowable capsule through the patient's gastrointestional tract to determine the location of target cells, such as cancer cells.

### BRIEF DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 is a diagram showing the various component portions of a system according to one embodiment of the present invention. The system can include a patient specific Detection Capsule 100, a Patient Data Collection Unit 200, Cell Marker Substance 300; a Physician Workstation 400 (such as located in the physician's office); and a centralized Data Collection and Analysis Center 500 located at a remote service provision site.

FIG. 2 is a schematic illustration of an exploded illustration of a Detection Capsule 100 according to one embodiment of the present invention. The capsule can include a coating 101; a pair of hemisphere end caps 102 (only one shown); a transmission module 120; with a transmitter 122; a RF antenna 124; a detector module 130; a preamplifier 131; a detector 132; a pulse-shaping amplifier 133; a detector electronics module 140; and a power connection means 150.

FIG. 3 is a schematic illustration of a flow diagram illustrating components useful according to one embodiment of the present invention for signal processing of radiation received by a Detection Capsule 100 with the solid-state detector based radiation detection embodiment. The components can include a solid-state detector 132; a preamplifier 131; a pulse-shaping amplifier 133; a plurality of Single Channel Analyzers 144; a control processor core 141; a write-once memory 143; a clock generator 142; a power control block 145; a communication link block 146; a transmitter 122; and an RF antenna 124.

FIG. 4 is the block diagram schematic illustration of a Patient Data Collection Unit 200 according to one embodiment of the present invention, including a receiver 201; control processor 202; write-once configuration memory 203; low-power data memory 204; serial data communication 205; user interface buffers 206; LCD or similar user interface display 207; membrane or similar keypad 207; and detachable serial communication cable 210.

FIG. 5 shows a capsule 100 and associated protective packaging 160 according to one embodiment of the present invention, including two package parts 160A and 160B and a magnetic structure 161 associated with at least one of the package parts.

FIG. 6 is a schematic illustration of one embodiment of a Physician Workstation 400 useful with the present invention. The Physician Workstation can comprise a workstation or personal computer 401 and a custom interface 402 including a receptacle 403 for receiving the capsule 100 enclosed in protective package 160; a receptacle 404 for receiving the marker vial 300; a built-in version of the patient data collection unit 405; and a socket 406 to accept the cable from or directly plug into a Patient Data Collection unit 200.

FIG. 7 is a schematic illustration of a graphical report which can be enerated according to one embodiment of the present invention, with position along the Gastro-Intestinal tract depicted along the horizontal axis, and a probability scoring depicted along the vertical axis, with Curve 450 depicting a normalized representation of the raw radiation counts per unit time, and Curve 460 depicting the probability (likelihood) score that a concentration of marker has formed at a position along the gastrointestinal tract.

FIG. 8 is a schematic illustration depicting a simulated normalized plot of radiation counts per unit time for a single detector with two collimator schemes, with Curve 2100 representing an uncollimated substantially isotropic detection response, and Curve 2102 representing a detector whose response pattern is substantially peaked in a radial fashion perpendicular to the major axis of capsule 100.

FIG. 9 is a schematic illustration showing relative performance of several detector schemes. The base embodiment of a single detector 2201; a two detector variation with 1 cm inter-detector spacing 2202; and a two detector variation with a 2 cm inter-detector spacing 2203.

FIG. 10 a schematic illustration showing dimensional features of a printed wiring assembly used to construct the capsule 100. The embodiment shows the extent of the battery 110; the insulating film 160; interconnection wires 170; and the encapsulant 101.

FIG. 11 shows the coordinate system used to discuss detector response patterns. The detector with a surface normal parallel to the z-axis is 2301, a random direction vector to a source is 2302, the projection of the direction vector on the XZ plane is 2303. The angle θ, known as the azimuth angle, is the angle from +z-axis to the projection 2303. The angle φ, known as the elevation angle, is the angle from the XZ plane to the direction vector 2302.

FIG. 12 shows the detector response of a typical Direct Detection (DD) radiation detector where the thickness of the detector is much less than the width or the height. Response in the azimuth and elevation directions is shown.

FIG. 13 shows the detector response of a typical Scintillator Detection (SD) radiation detector where the scintillation crystal is a unit cube.

FIG. 14 shows the detection efficiency (number of events captured per incident event) of a typical Direct Detection (DD) radiation detector. Note that detection efficiency is a function of detector thickness.

FIG. 15 shows a stack of Direct Detection (DD) radiation detectors. Note that the detectors, 2401 and detectors 2402 need not be of the same physical dimension. Note that a flexible or conformal circuit such as 2403 can be used to interconnect devices.

FIG. 16 shows the geometric arrangement of a collimator.

FIG. 17 shows the effect of changing j in the simplified model used to predict collimator response.

FIG. 18 shows a forward-looking collimator for a DD system.

FIG. 19 shows a side-looking or radial collimator for a DD system.

FIG. 20 shows a skew collimator for a DD system.

FIG. 21 shows a typical Charge Amplifer.

FIG. 22 shows the transfer function and operation of a typical Pulse Shape Amplifier.

FIG. 23 shows a typical Analog Single Channel Analyzer.

FIG. 24 shows a typical Digital Single Channel Analyzer.

### DETAILED DESCRIPTION OF THE INVENTION

The present invention provides medical devices and methods for detecting abnormal tissue, such as cancerous tissue. The invention is especially applicable for use in detecting cancer of the gastrointestinal tract (GIT). While the present invention is described with respect to use with a human patient, it will be understood that the present invention is applicable for use with non-human patients.

### Method

In one embodiment, the present invention provides a method for locating abnormal tissue growth, such as cancer. The method can include the steps of providing a material having an affinity for a target tissue type, such as cancer, and a capability for providing a detectable signal, such as the cell marker (CM) 300; administering the material to the patient; providing an swallowable pill or capsule, such as the Detector Capsule (DC) 100 having a detector for receiving a signal emitted by the material; directing the capsule with detector through at least a portion of the patient's gastrointestinal tract (GIT); providing a means to communicate said received signals to a data collection device, such as the Patient Data Unit (PDU) 200 having a data communication link with the DC and a means for storage of said data; providing a means to analyze said data, such as the Data Processing Center (DPC) 500 having a means to gather said data from a plurality of PDUs and to organize said data into human readable form; and providing a human interface for management of the method and display of said human readable form of the data, such as the Physicians Workstation (PWS) 400 enabling a skilled observer to determine the presence and location of cancerous material.

By giving the patient certain materials that have high affinity to the cancer, and that also emit a certain signal as they bind to that tissue, the observer can note if and where the signal is coming from. In this approach, once can first identify some part of the cancer cell that stands out as different than normal cells, and then to construct a specific marker that will identify this moiety and not “innocent bystander” cells that are normal. Such a differentiating feature is often called “tumor associated antigen”. This name makes the point that this antigen (protein) is associated only or at least overwhelmingly with cancer cells, while it is substantially absent from normal cells.

In normal radiolabeled radiation imaging systems, such as a Gamma Camera or SPECT imager, a collimator is used to provide a highly directional “ray” emanating from a constrained physical region (2-dimensional: “pixel”; 3-dimensional “voxel”) of the object being imaged by intercepting thousands of “rays” and relating them to an associated pixel (Gamma Camera) or voxel (SPECT imager).

In the applications addressed by this invention, information regarding the distribution of marker material is determined by proximity of the radiation source to the detector. Specifically, the distributed marker sources are isotropic radiators and therefore the radiation flux at any distance r from the source is proportional to the square of the distance in a form such as

Iⱼ(r)=Iⱼ₀/r²

Devices

Materials for Binding and Marking:

Materials useful in the present invention include a signal emitting substance (a “marker”) such as a radioactive substance, magnetic substance, fluorescent substance, or ultrasonic contrasting agent in combination with one or more substances that bind preferably to cancer cells, while normal tissue is substantially not bound (a “differentiator”). In one embodiment, a suitable material can comprise one or more radioactive markers in combination with a protein or protein complex differentiator that has an affinity for a particular target cell type.

A suitable marker can include one or more radioactive nuclides. Radioactive nuclides useful in the present invention are those that emit gamma radiation and whose stable isotope is biologically acceptable. In some applications it can be desirable for a radioactive marker to have a half-life comparable to or longer than the nominal transit time of ingested material through the subject gastrointestinal system. It can also be desirable to use an entity that emits gamma radiation low enough to be efficiently collected in detection devices (less than about 1MeV). Suitable radioactive isotopes include but are not limited to ⁴⁸Cr, ^{99m}Tc, ⁶⁴Cu, ¹⁵³Dy, ¹⁵⁵Dy, ¹⁵⁷Dy, ¹⁸⁸Ir, ⁵²Fe, ³⁸K, ⁸³SR, ¹²²Xe, ²⁵Xe, ⁸⁷Y, ⁶⁶Ga ²⁰¹Tl, ¹¹¹In, and ¹⁰⁹In. In one embodiment the marker is ^{99m}Tc, the metastable isotope of the element Technetium, that decays by emitting a single gamma particle at 143 keV with a half-life of 6.01 hours.

A suitable differentiator can be one or more monoclonal antibodies (MAb).

Monoclonal antibodies useful in the present invention include, but are not limited to those that have an affinity for the TAG-72 protien such as the commercial product Oncoscint® (Cytogen Corporation), the carcinoembryonic antigen (CEA) such as the commercial product CEA-scan® (Immunomedics®, Inc.) or other proteins associated with colorectal cancer such as 17-1A.

The following documents/information are incorporated herein by reference in their entirety: “Clinical and Technical Considerations for Imaging Colorectal Cancers with Technetium-99m-labeled AntiCEA Fab Fragment” by Deborah A. Erb and Hani A. Nabi of Dept of Nuclear Medicine, SUNY at Buffalo N.Y., Journal of Nuclear Medicine Technology, Volume 28, Number 1, March 2000; “Indium-111 Satumomab Pendetide: The first FDA Approved Monoclonal Antibody for Tumor Imaging” by Paul J. Bohdiewicz, Nuclear Medicine Dept. William Beaumont Hospital, Royal Oak, Mich., Journal of Nuclear Medicine Technology, Volume 26, Number 3, September 1998.

In an alternative embodiment, the differentiator can be selected from a group including peptides and nucleotides. Specific examples of each class are beginning to appear in academic papers with no commercial embodiments at this time. Peptides and nucleotides behave similarly to the MAb technology previously described.

In a further alternative embodiment, the marker can be a nano-particle. Nano-particles are inorganic materials that are conjugated to MAb, peptides or nucleotides in a similar fashion to the previously described radioactive marker.

In an alternative embodiment, other substances can be used in addition to or in place of the monoclonal antibodies for carrying or otherwise directing a substance to targeted cells or organs. For instance, a substance comprising an aqueous core and one or more outer layers (including lipid containing layers such as phospholipid layers) can be used for conveying a radioactive material to a target cell or organ. A suitable substance includes one or more liposomes. The term Liposome, as used herein, refers to an artificial microscopic vesicle having an aqueous core enclosed in one or more phospholipid layers, used to convey a substance such as vaccines, drugs, radioactive materials, enzymes, or other substances to target cells or organs. Suitable commercially available liposomes include Abelcet®, which is Amphotericin B, manufactured by The Liposome Company, Inc., One Research Way, Princeton, N.J. 08540-6619, and Doxil®, which is Doxorubicin, manufactured by ALZA Corporation, 1900 Charleston Rd., Mountain View, Calif. 94039-7210.

According to one embodiment of the present invention, and in the description set forth below, the cell differentiator substance 300 can include a material comprising, in combination, a differentiator such as an MAb and a marker such as ^{99m}Tc.

Hardware & Software

Capsule:

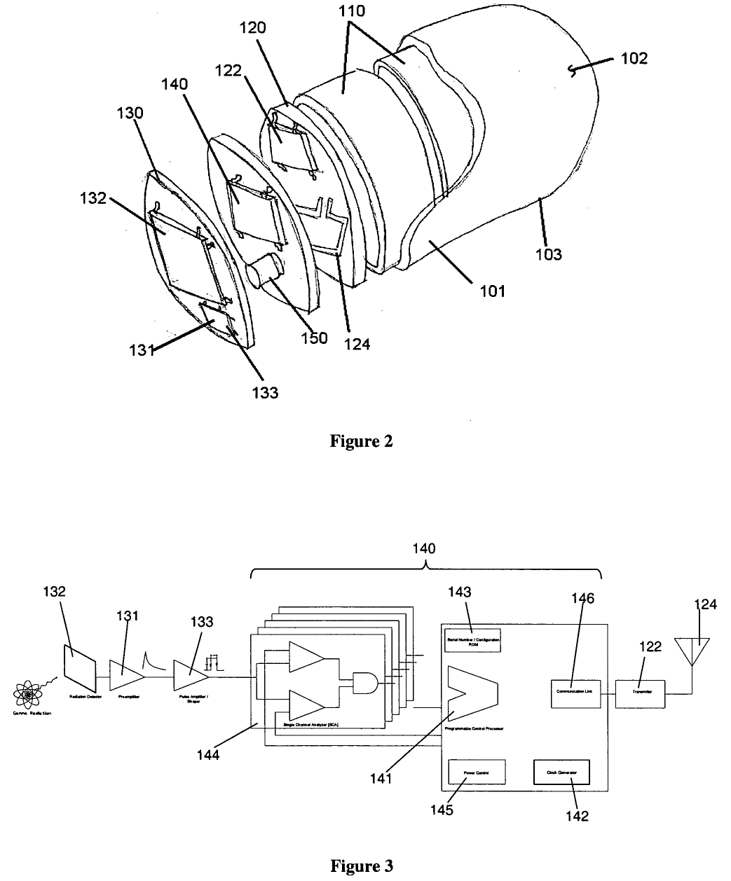

Referring to FIG. 2, in one embodiment of the present invention, a capsule 100 adapted for swallowing by the patient is provided with a detector 132, which can be mounted on a detector module 130 supported in the capsule 100. The detector is capable of detecting the signal emitted by the marker. Because the marker is associated selectively with cancerous cells (or other target tissue cells) via the differentiator substance, the locally dense concentration of the differentiator in cancerous tissue cells will be detected by the detector on board the capsule as it passes in close proximity to the cancerous tissue.

Upon ingestion, the capsule travels through the gastrointestinal tract, such as by normal peristalsis. The signal may be transmitted by the capsule immediately to a receiver or Patient Data Unit (PDU) outside or inside the body, or recorded for future interpretation. For instance, the PDU can comprise a device that can be supported on the patient's wrist or otherwise associated with the patient's body or clothing during the time the capsule 100 is passing through the GIT. The capsule is later excreted in the stool in the normal fashion, and can be retrieved if necessary. By traveling along the gastrointestinal tract within the capsule, the detector is in close proximity to tissues of the esophagus, stomach, small bowel, colon and rectum. This proximity can provide improved sensitivity and specificity compared to traditional external gamma radiation detection and imaging means such as Gamma Cameras and SPECT imagers and allow for the detection of small pre-cancerous and cancerous lesions that might otherwise escape detection. Furthermore, this device may also sense signals coming from non-contiguous but close structures, including the pancreas, kidneys, spleen, bile ducts, gallbladder, liver and the genito-urinary system.

The capsule 100 can comprise any detector 132 suitable for detecting the presence of the marker substance administered to the patient. Suitable detectors include but are not limited to ionizing radiation detectors or magnetic particle detectors. Ionizing radiation detectors could be based on solid-state direct radiation detectors or photo-detectors with attached scintillation crystals. Magnetic particle detectors could be based on sensitive magnetometers or reluctance meters. Alternatively, a detector module can be located on a flexible endoscope, such as on a colonoscope or a sigmoidoscope.

The capsule 100 can also include one or more power source, such one or more battery modules 110. Alternatively, the capsule 100 can receive power via a radio frequency (RF) power source. The capsule can also include a transmitter 122 associated with a transmission module 120 for sending raw or processed signal data received by the detector to the receiver 201 or other remote location outside the patient's body, and/or a recorder for recording the signal received by the detector. The receiver 201 outside the patient's body can be adapted to receive and/or record the signal sent from the capsule.

Capsule 100 can have an outer surface 101 which is shaped to aid in ingesting the capsule, and can include a plurality of coatings, one of which is a protective coating which is acid tolerant. Other organic and inorganic coatings can be applied. By example, coating the surface with Manganese dioxide (MnO₂) may create a laxative effect resulting in more rapid passage of the capsule through the tract. Coating the surface with a diuretic such as loop diuretics (e.g. bumetanides, furosemide), thiazide diuretics (e.g. hydrochlorothizide, chlorozide and chloralidone) and potassium sparing diuretics (e.g. amiloridetramterene) will cause accelerated elimination of unassociated markers in the kidney and urinary tract.

Alternately, the desired biological effects listed above can be obtained in the normal fashion (i.e. by oral methods) rather than as a coating on the capsule 100.

As shown in FIG. 2, capsule 100 can have a generally hemispherically shaped end cap 102, though other smooth tapered shapes can also be employed. In FIG. 2, only one generally hemispherically shaped cap is shown, though it will be understood that such a shaped cap 102 can be disposed on one or both ends of the capsule 100.

The capsule 100 can include one or more battery modules 110 for providing on board power or energy. The capsule can also include a transmission module 120 including a RF antenna 124 and a digital RF transmission circuit 122 and on board digital support, control and logic circuits 125 powered by the on board battery. In the preferred embodiment the transmission module components 122 and 124 comprises an active RF transmitter, meaning that the communication function is achieved by supplying radiating energy from an on board power source. In an alternative embodiment the transmission module components 122 and 124 comprises a passive or “zero-power” RF transmitter, meaning that the communication function is achieved by altering the apparent RF load seen by a remote RF transmitting power source. In this embodiment, the remote RF power source can also provide a portion of or all of the on board power requirements reducing of eliminating the need for energy supplied by battery modules 110.

In one embodiment, the power source is a battery 110 selected for energy density and discharge characteristics. One suitable battery chemistry is Silver-Oxide as represented by the Duracell D357 coin cell battery.

The transmission module 120 is selected for efficient short-range unlicensed operation. Low-power implementations of the transmitters 122 incorporated in the Bluetooth® or IEEE 802.1 lb standards provided, for example, in the Agilent Technologies E8874A Wireless LAN Design Library that can be incorporated into a single purpose radio frequency integrated circuit or as part of an Application Specific Integrated Circuit (ASIC) are preferred. In an alternative embodiment, a custom protocol optimized to transmit energy minimization and low data rate communication can be used. The antenna 124 is custom designed to complement the characteristics of the chosen transmitter and the physical constraints of the capsule.

The capsule 100 can include a detector module 130 comprising a suitable detector 132, preamplifier 131, and a pulse-shaping amplifier 133. The detector is preferably a solid state radiation detector when a radioactive marker is employed. The detector module 130 should have adequate dynamic response to allow unambiguous collection of high and low count-rate gamma events. High count-rate gamma events arise from unbound markers circulating in the patient's blood pool and temporarily resident in various non-cancerous tissues as a result thereof. Low count-rate gamma events arise from the plurality of cancerous tissue source. A 1000:1 count rate differential between High and Low count conditions may be encountered.

Solid-state radiation detection devices and methodologies are preferred in one embodiment of the present invention. Alternatively, detector 132 can be a solid-state scintillation detector comprised of a solid-state photo-detector (such as the Detection Technologies PDB or PDC series) coupled to a scintillation crystal to convert the gamma event to a number of photons. A lower count threshold can be representative of a 1-50 nano-Curie source and the detector module 130 can be adapted to accommodate this level of activity.

Referring to FIG. 3, the preamplifier 131 can be used to convert charges created in direct solid-state detection devices or current generated in the photo-diode of a scintillation detection devices into a voltage output. The output voltage magnitude is proportional to the energy of the gamma radiation incident on the detector 132. The pulse shape of the output can be determined by various circuit elements.

Pulse shaping amplifier 133 accepts the output of charge preamplifier 131 converting it to an output voltage pulse. The amplitude of the output pulse can be linearly related to the magnitude of the input signal. The pulse shape can be substantially rectangular with a predefined and constant width “w” and a variable height “h” depending on the incident energy of the particles impacting the detector.

The capsule can include a detector electronics module 140. The module 140 can include detector support electronics and a control processor. In one embodiment, an Application Specific Integrated Circuit (ASIC) that contains a programmable control processor 141, a clock generation and timing module 142, a write-once configuration memory 143, a plurality of single channel analyzer modules 144, a power control module 145 and a communication link module 146 can be employed.

The preferred programmable control processor 141 is based on a common commercial microcontroller core such as one based on the Intel 8051 8-bit processor instruction set and architecture. Instructions governing the operation of the capsule are stored in the read-only memory embedded in the microcontroller core module. The microcontroller core can also be responsible for the management, control and data transfer between all portions of the ASIC and attached components.

The clock generation and timing module 142 can be responsible with generation of all internal clock signals required on the ASIC.

A write-once configuration memory 143 can be provided to retain personalization information for the capsule. At manufacture, a unique serial number and various hardware/software configuration parameters can be loaded. These parameters can be read by the programmable control processor 141 as often and frequent as necessary for proper operation of the capsule. The unique serial number can be used to identify the capsule to the receiver system to facilitate correlation of test results to patients. Alternatively, a unique serial number or other identifier can be associated with the capsule by other methods, such as by a magnetic or optical tag or indicia, to correlate the capsule and test results to a particular patient.

At least one single channel analyzer (SCA) 144 can be provided, and in one embodiment a plurality of SCAs 144 is provided to interpret the output of the pulse-shaping amplifier 133.

Internally, the SCA can include two analog magnitude comparators and logic circuits to create an output pulse each time a voltage below, between, or above a predetermined range or value is received. For instance, an output pulse can be created each time a voltage between or possibly equal to the programmed values of the magnitude comparators occurs. To allow calibration at manufacture, the high and low limit setpoints, while analog in nature, can be determined by digital to analog converter circuits whose digital program values are stored in the write-once configuration memory 143. The output of the each SCA 144 is provided to and accessible by the programmable control module 141. Alternatively, the SCA can be substantially digital in nature by using a single initial analog-to-digital converter (ADC) to convert the input pulse height into a digital signal value. The magnitude comparison function described above can be replaced by a digital comparison function where the calibrated low and high limit setpoints are determined at manufacture and stored in the write-once configuration memory 143.

The power control module 145 is used to manage power to some or all portions of the capsule. The module 145 can be used to conserve battery power through various load management schemes including, but not limited, to activating and deactivating various electrical modules such as the preamplifier, pulse-shaping amplifier and transmitter.

The communication link module 146 accepts digital data words from the programmable control processor and formats them for correct transmission via the transmitter 122.

The capsule can also include a power connection means 150. In one embodiment, the power connection means is a magnetic reed switch that is in series with the battery 110 and the remainder of the capsule electronics modules. Alternatively, active switches such as one based on a Hall-effect sensor can be applied. Choice of switch means is based on current carrying capacity and shelf life requirements. In operation, the power connection means 150 is “open” or in the disconnected state when a appropriately poled magnetic field is placed in proximity to the switch. When the magnetic field is removed from the proximity of the switch or an opposing field is provided to cancel the first field, the power connection means 150 is “closed” or in the connected state. When the power connection means is in the “closed” state, the capsule is operational.

Referring to FIG. 5, the capsule can be enclosed in a protective package 160. The protective package provides protection from physical abuse and from various environmental contaminants (e.g. dust, moisture, and bacteria). According to one embodiment, a magnet can be included in the protective package, wherein the magnet is appropriately poled and positioned to maintain the power connection means 150 in the “open” state when the capsule is contained within the protective package 160. When the patient removes the capsule from the protective package 160 prior to ingestion, the power connection means 150 is released to the “closed” state and the capsule electronics is activated. As shown in FIG. 5, a magnetic structure 161 can be associated with one of the package parts 160A/160B such that when the package parts are separated to open the package and remove the capsule, the power connection means is released to the closed state. Alternatively, other methods of activating capsule power can be used, including without limitation mechanical activation (such as with mechanical switches or materials that are moved, removed, or articulated when the package is opened), light or optical activation, vacuum or air pressure activation, and the like.

Radiation Detecting Capsule

One embodiment of the detection capsule 100 is a radiation detection capsule. This capsule is used with a radiolabeled differentiator.

Construction Methods

One method of construction useful for manufacture of the capsule is a “stacked hybrid” approach. In this approach the various electronics-based portions of the detector capsule are each constructed on a printed wiring assembly (PWA) configured in a generally circular planar format. Non-electronics based portions (i.e. a battery 110) can be included. Each PWA can provide a circuit layer in the “stacked hybrid” configuration with appropriate circuitry applied to each. Connection between PWA circuit layers can be accomplished by soldering non-insulating wires in slots on the periphery of the PWA. Connection between a PWA circuit layer and a non-PWA layer can be accomplished via a pressure contacting arrangement (e.g. the central electrode contact of the Duracell D357 battery and a mechanically matching conductive pad on the facing surface of the adjoining PWA).

Subassemblies

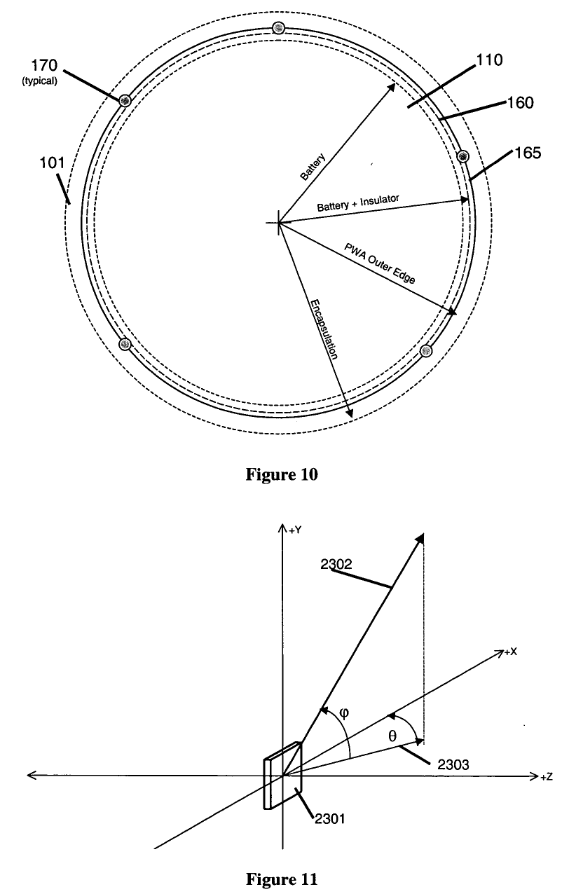

The diameter of the PWA (see FIG. 10) may be determined by the diameter of the battery 110. Specifically, the outer diameter of the PWA can be the diameter of the battery 110 (e.g. 11.6mm diameter for the Duracell D357) plus twice the thickness of an electrical insulation film 160 (e.g. 0.02 mm thick mylar) plus the diameter of a small non-insulated electrical wire 170 (e.g. 0.125 mm for a 36 ga wire). The total outside diameter of the PWA based on the above example is less than 12 mm, and is about 11.77 mm. Material selection for the PWA is based on anticipated environmental factors and interconnection complexity. One embodiment can include a 1.25 mm thick FR4 copper-clad laminate. Design and assembly of the PWA can be accomplished using standard “chip-on-board” or hybrid packaging tools and equipment.

Alternatives to the round PWA configuration include various non-circular shapes, including without limitation polygonal and oblong shapes. Using polygons of order 4 (i.e. a rectangle), order 6 (i.e. a hexagon), or 8 (i.e. an octagon) can lower the cost of PWA fabrication. The polygon would be inscribed in the circular extent of the PWA 165. Interconnection between PWA circuit layers can be accomplished by a single non-insulated wire at the vertices of the polygons or a number of wires at or nearby the vertices of the polygons. The order of the polygon used can be determined by analyzing the interconnect pattern between PWA circuit layers.

Encapsulation

Once all of the component layers of the “stacked hybrid” are assembled, the entire assembly can then be inserted into an encapsulation medium such as epoxy or gelatin via an injection molding or other manufacturing process.

The encapsulation material is chosen from that class of materials that is approved for ingestion, is completely or largely immune to attack by gastric and intestinal secretions. The chosen material must have a working viscosity consistent with the molding process and must not create a surface chemistry problem with the PWAs or other internal components.

Coatings

Subsequent to or in conjunction with the molding step, a bio-available compound can be included. If the encapsulation material provides a biodegradable component, this material can be included in the encapsulant material to provide a delayed release. If the encapsulation material is inert, then a delay or immediate release coating can be applied to the exterior surface of the capsule after encapsulation.

Detector

Radiation detectors are available in a number of types and configurations, and can be categorized in groups, such as the groups of Direct Detectors (DD) and Scintillation Detectors (SD).

Suitable solid state detectors 132 can include, without limitation to type, one or more of the following (e.g. High Purity Silicon (HPSi) such as the Detection Technologies XRA or XRB series; Cadmium Telluride (CdTe); Cadmium Zinc Telluride (CdZnTe)1; High Purity Germanium (HPGe) or Mercuric Iodide (HgI₂). The High Purity Silicon Detectors (HPSi) class such as the Detection Technologies XRA or XRB series exemplifies the DD group.

The SD group is exemplified by the Thallium activated Cesium Iodide (CsI:Tl) scintillation material coupled with a high efficiency photodiode such as the Detection Technologies PDB or PDC series. Suitable scintillation material can be, without limitation to type, one or more of the following examples: Cesium Iodide (CsI), Cesium Iodide with Thallium activation (CsI:Tl), Cesium Fluoride with Europium activation (CsF:Eu), Bismuth Germanate (BGO), Lutetium Oxyorthosilicate with Ce³⁺activation, Yttrium Aluminum Garnet with Cerium activation (YAG:Ce), Yttrium Aluminum Perocskit with Cerium activation (YAP:Ce), Sodium Iodide (NaI), or Sodium Iodide with Thallium activation (NaI:Tl).

Signals from the DD group can be easier to acquire and analyze than are those from the SD group. However, the collection efficiency of the DD group is only a few percent while it is nearly 100% for most configurations common to the SD group.

Inherent in any detection scheme is the notion of directional sensitivity or the probability that a single nuclear particle (such as gamma particles) arriving from a specific direction will be captured by the detector. Once captured a DD or SD creates an output proportional to the incident energy of the particle. FIG. 11 shows the coordinate system to be used in conjunction with the following descriptions.

Direct Detection Group (DD)

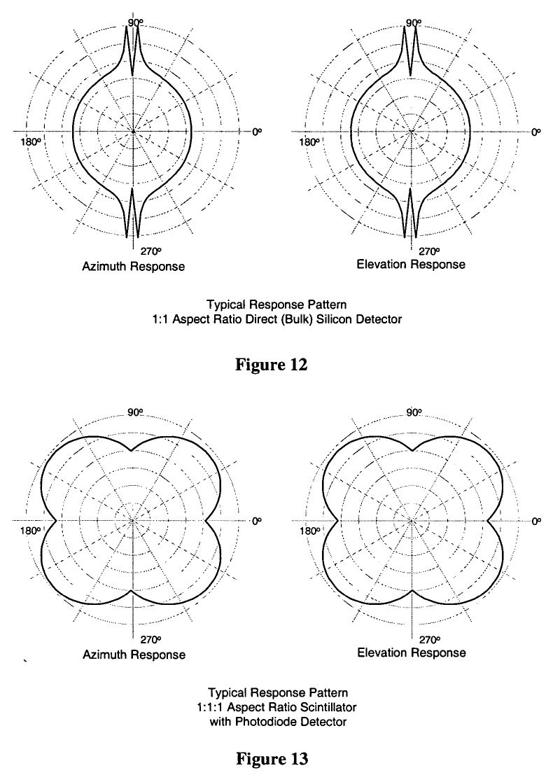

FIG. 12 shows a typical response pattern for the DD group. The response is a linear function of the straight-line path created by the intersection of the particle's path with the included volume of the detector. In this figure, the detector is assumed to be of equal length in the x and y axis with a much smaller thickness in the z dimension. Typical DD devices (e.g. the Detection Technology XRB series) that might fit within the capsule 100 have x and y dimensions of 5 mm with a thickness or z dimension of 0.3 mm. While a first approximation to the elevation response can be made using the physical thickness of the detector, the actual depletion region of the device (as determined by an applied bias voltage) would more accurately reflect the physical situation. In practice, bias voltages are chosen such that the maximum depletion region depth is achieved. That depth is nearly the total thickness of the device. The response pattern can be symmetrical about the XY plane despite the non-symmetric nature of the physical detector.

Stacking Detectors

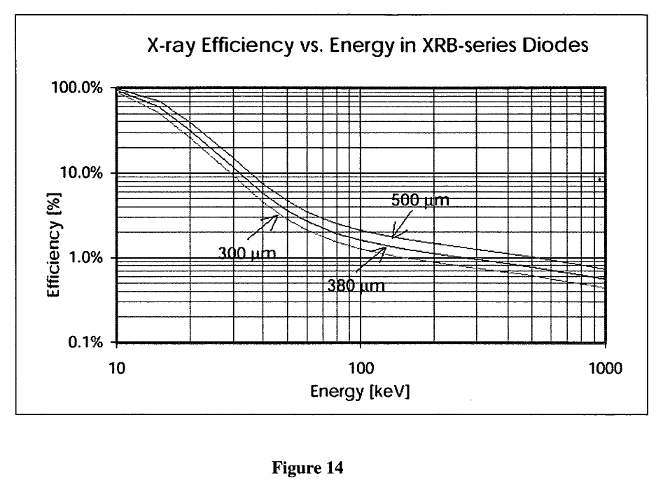

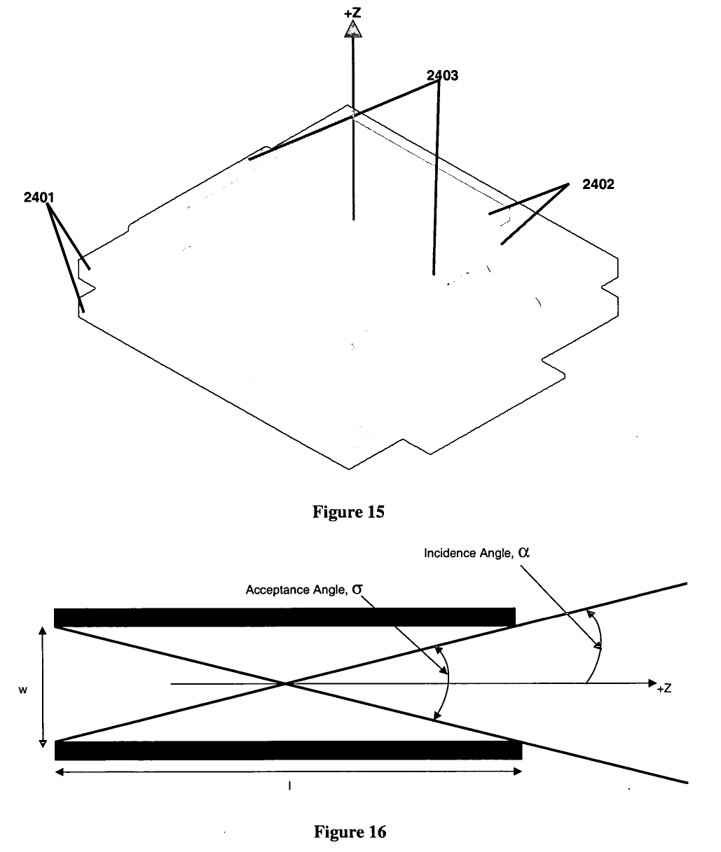

While the DD group provides simple electrical interfacing, it may be less efficient (ratio of detected gamma per incident gamma) than is desired for a particular application. At the energy level of the ^{99m}Tc gamma, a typical DD device has a detection efficiency of about 1.5% as shown in FIG. 14. As shown in FIG. 14, detection efficiency improves as the thickness of the detector increases. However, it is not feasible to increase the thickness of a DD device without bound. To approximate a thick DD detector, it is possible to stack detectors as shown in FIG. 15. In this figure, two similar size detectors 2301 are shown with two smaller but similar size detectors 2302 stacked to form a detector four times as thick as a single detector, in it's central region (along the Z-axis). In this type of arrangement, all the detectors can have their diode junctions connected in an electrically parallel circuit.

As is shown in the diagram of FIG. 15, multiple physical detector sizes can be stacked. If the lateral dimension (in the XY plane) is chosen properly, a stacked detector substantially filling the hemispherical end cap 102 can be constructed thereby optimizing the detection efficiency of the DD scheme.

By connecting each of the stacked detectors in parallel with all of the other detectors, a single low-noise charge amplifier can be used in order to reduce the energy consumption of the detector module 130. Additionally, this type of arrangement reduces the thermally induced noise common to charge-based detectors called 1/kTC noise by reducing the effective capacitance of the detector. In this application, k is the Boltzman's constant, T is the temperature in degrees Kelvin and C is the effective capacitance of the charge storage/generation device.

Collimating

A collimator can be used to provide additional directionality to a detector response curve. For gamma radiation, collimators can be made from a high-Z (atomic mass) material such as Lead (Pb). In construction, a collimator resembles a pipe with a large I/w (length/width (or diameter)) ratio. For simple calculations, the effect of a collimator is to eliminate all gamma radiation that attempts to strike the detector at an angle greater than the acceptance angle (σ) of the collimator. To the first approximation, this effect can be modeled as a cosine function of the form

i_{c}=i cos ^{j}(kα)

where i_{c} is the intensity of the beam at the detector end of the colimator, i is the intensity of the beam entering the collimator at an angle α and j,k are constants based on the l/w of the collimator. Referring to FIG. 16, the acceptance angle is defined by an equation of the form

σ=2 tan⁻¹ (w/l)

Since a beam has zero intensity with an incident angle of α and the nature of the cosine function providing zeros values at ±π/2 radians. Then in radians, k can be given by:

k=π/σ

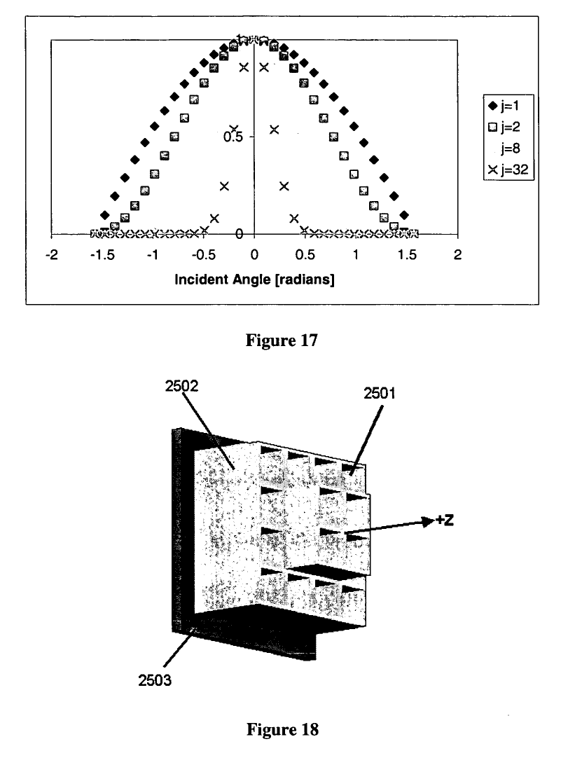

Depending on the material choice for the collimator and the l/w chosen, various values of j are possible. The higher the value of j the more rapid the increase in attenuation of the incident beam as |α| increases. This relationship is shown in FIG. 17.

In this invention, the nominally omni-directional response of an ideal detector can provide significant advantages. For some applications (e.g. locally concentrated background interference from unbound differentiator material), it might be desirable to tailor the response pattern of the DD detection scheme. This is accomplished through the use of collimators or unique combinations of basic DD devices.

Unlike in a standard gamma-imaging device, when a collimator is used, poor collimator efficiency (i.e. large acceptance angle) is acceptable as only a small off-axis rejection can eliminate many sources of background interference. In a standard gamma imaging device, there a no intense background interference sources from 90 through 180 degrees (see FIG. 12) and a collimator located in the +Z direction is all that is required. In this invention, there are intense background interference sources in all directions and therefore, two identical collimators can be used: one each for the +Z and −Z directions.

Forward Collimator

A weak forward facing (+Z) collimator is easy to implement with a grid of 4 to 16 collimator “holes” as shown in FIG. 18. Note that in this figure, +Z elevation was given to the central 4 collimator “holes” 2501 that make-up the 16 “hole” collimator array 2502 located on the +Z side of the detector 2503. This elevation can be used to provide even greater +Z directivity. The elevation could also be used to allow the collimator to more closely approximate the shape of the end-caps 102.

Radial Collimator

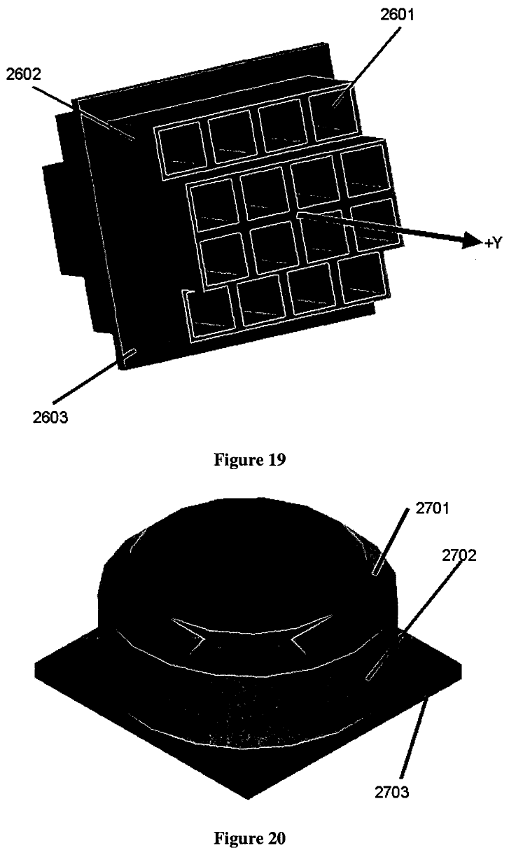

A weak radial facing (+Y) collimator can be implemented with a grid of 4 to 16 collimator “holes” as shown in FIG. 19. Note that in this figure, +Y elevation was given to the central 8 collimator “holes” 2601 that make-up the 16 “hole” collimator array 2602 located on the +Y side of the detector 2503. This elevation can be used to provide even greater +Y directivity. The elevation can be be used to allow the collimator to more closely approximate the shape of the capsule 100. In this figure, two collimators are shown—one on each side of the detector. If this configuration is used with a detector whose normal is directed perpendicular to the axis of the capsule 100, then there can be two narrow acceptance slots.

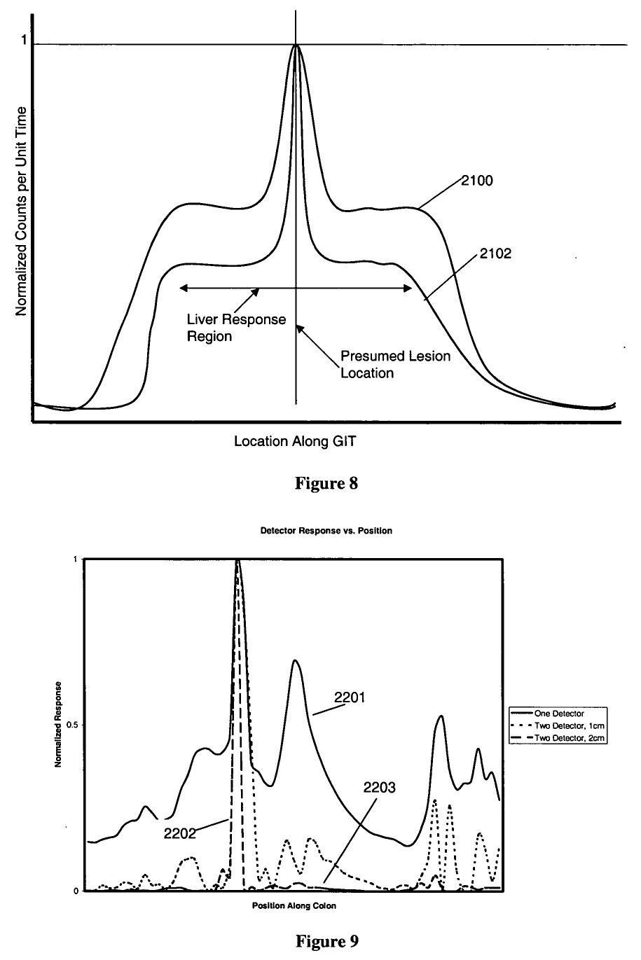

FIG. 8 provides a schematic illustration depicting a simulated normalized plot of radiation counts per unit time for a single detector with two collimator schemes. Curve 2100 represents an uncollimated substantially isotropic detection response, and Curve 2102 representing a detector whose response pattern is substantially peaked in a radial fashion perpendicular to the major axis of capsule 100.

Skew Collimator

A weak collimator that accepts radiation preferentially in a forward facing angle such as 45 degrees off the +Z axis can be provided as shown in FIG. 20. If a shield is placed in the −Z axis direction, then the response will be maximum for a ring-like region symmetrical about the +Z axis and at an angle of 45 degrees.

Scintillation Detection Group (SD)

FIG. 13 shows a typical response pattern for the SD group. The response is governed by the projection of the extent of the scintillation crystal faces onto the sphere centered at the source and intersecting the centroid of the scintillation crystal. In this figure, the detector and scintillation crystal are assumed to be of equal length in the x and y-axis. In this example the thickness (z-axis extent) is assumed to be of the same length as the x and y-axis extents. A suitable SD device can be a combination of a CsI:Tl scintillation crystal tightly coupled to a high efficiency photo-diode(e.g. the Detection Technology PDB series) positioned within the capsule 100, with x and y dimensions of 5 mm and a thickness or z dimension of 5 mm. It should be noted that the response pattern is symmetrical about the XY plane.

Collimating

Collimating for a SD detector can be provided in provided in traditional gamma camera and/or SPECT imaging devices. Referring to FIG. 14, altering the 3D-aspect ratio of the scintillator can effectively tailor the response pattern of the SD device. Altering the Z axis dimension of the scintillator crystal can affect the sensitivity in the XY plane. For example, increasing the Z-axis dimension will increase the relative sensitivity in the direction perpendicular to the Z-axis.

Two Detector Configuration

In one embodiment, the capsule can comprise a plurality of solid-state radiation detectors associated with the detector module 130. For instance, in a two-detector system, first and second detectors can be disposed at opposite ends of the capsule. Each detector may or may not have associated with it a collimator device. The collimator device restricts the solid angle through which the detector can sense incoming gamma particles. An isotropic detection pattern is one in which there is not particular direction and solid angle in which the detector is more or less sensitive than in any other direction and solid angle. FIG. 9 shows the simulated response of a two-detector system with two inter-detector spacings (1 cm and 2 cm). The response from each detector can be separately utilized, or alternatively, a “system response” can be provided as the difference response of the two detectors for each sampling period. The difference between the responses of the two detectors (subtracting one from the other) can be useful in determining directionality of a source of signal, and for eliminating background signal noise. Other combinations of multiple detector responses (eg addition, multiplication, integration, differentation) are also possible.

Position Tracking

During the course of travel through the GI tract, the capsule may experience forward motion, retrograde motion, and tumbling. Accordingly, it may be desirable to provide a device for determining and/or tracking the position of the capsule in the GI tract. For instance, electrical, electromagnetic, magnetic, or radioactive signals can be used with multiple receivers and triangulation methods to assist in locating the capsule. For instance, a multiplicity of radiolabeled markers at known locations internal or external to the body can be detected by the detector within the capsule to establish the capsules position and orientation with respect to the known locations. Alternatively, the capsule can include an inertial position sensing system, such as a system of one or more accelerometers for detecting and tracking the capsules position and orientation in the GI tract. For instance, in one embodiment, the capsule can be provided with a three axis accelermoter, and a data receiver worn by the patient can include a three axis accelerometer.

A data receiver can be worn on the trunk of the patient's body and the data receiver can be equipped with an accelerometer. It can be desirable to know the position and orientation of the capsule each time an integrated radiation count is reported compared to the position of the capsule at the previous time a radiation count is reported. The position and orientation of the capsule can be tracked with position measurements obtained from an accelerometer mounted on the patient (to take into account the gross motion of the patient). Integration of the motions can be used to track position and orientation of the capsule between the time the capsule is swallowed and the time the capsule is eliminated from the patient's body.

Detector Readout Electronics

Detector readout electronics can multiple blocks. Referring to FIG. 3, a functional block in direct communication with the detector, either of the DD of SD type, is a charge amplifier 131 which is followed by a shaping amplifier 133.

Charge Amplifier

The charge amplifier 131 can be used to detect small quantities of electric charge created by direct detection of gamma rays (DD) or photons (SD) at the time of a gamma capture event. The charge amplifier can provide an output signal that is proportional to the energy contained in the incident gamma ray. Since it is anticipated that the number of gamma events per second encountered in certain regions of the GIT will be extremely low, it can be useful to provide a charge amplifier that exhibits relatively low electrical noise characteristic. The following reference is incorporated herein by reference for teachings regarding noise sources and control of those sources in a charge amplifier: Radeka, V; “Low noise techniques in detectors”, Ann. Rev. Part. Sci. 38, p.217 (1988).

It can be desirable to achieve low electrical noise performance by limiting the bandwidth of the amplifier. It can also be desirable to preserve at least a 1000:1 pulse rate capability for detection of target tissue according to the present invention.

In one embodiment of the present invention, a lower limit on event rate is based on a resolution of about 0.1 μCi. For a ^{99m}Tc source and a DD detector, the resulting current can be about 25 pA based on approximately 3.7×10³ captures where each individual capture would result in a charge of approximately 6.84×10⁻¹⁵ coulombs. At an upper end, event rates on the order of 3.7×10⁶ captures per second can be anticipated. Accordingly, to create a signal to noise ratio of 6 db, and to provide adequate time resolution to prevent “pulse stacking”, total input referred noise current could be less than 6 pA with a bandwidth of about least 8 Mhz.

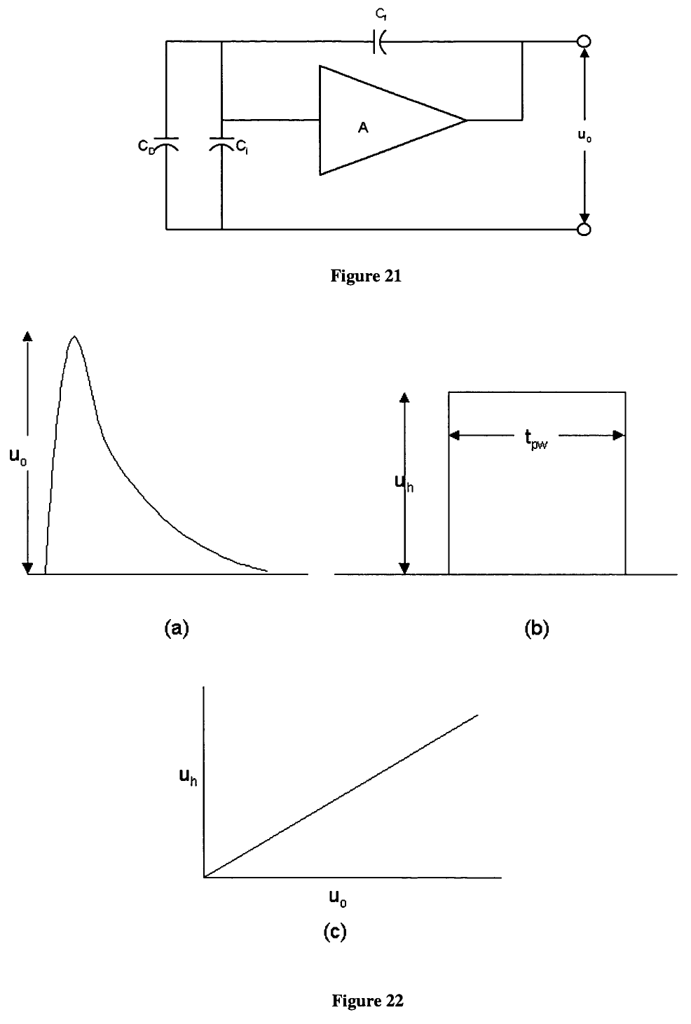

A simplified schematic diagram of one possible embodiment is shown in FIG. 21. For more details on various design aspects of charge amplifiers, one can refer to one of a number of references, including “Semiconductor Radiation Detectors” by Dr. Gerhardt Lutz published by Springer-Verlag, which reference is incorporated herein by reference.

A possible embodiment for this amplifier can be a single high gain transistor of the JFET type mounted on the same circuit card as the detector and as close to the detector as terminals as possible to minimize Cᵢ. An other possible embodiment for this amplifier can be a single DEPFET integrated into the DD or SD solid-state device with the feedback capacitor, C_{f} disposed on the same semiconductor die or on the circuit card immediately adjacent to the semiconductor mounting location. The DEPFET structure can be adapted to operation as an active amplification device integrated into a high purity silicon wafers used to create high performance DD devices and the photo-detectors that are included in the SD device. For a description of the DEPFET structure and operational parameters, the following reference is incorporated herein by reference: “Semiconductor Radiation Detectors” by Dr. Gerhardt Lutz published by Springer-Verlag

Pulse Shaping Amplifier

The output of the Charge Amplifier can be applied to the Pulse Shaping Amplifier (PSA). As shown in FIG. 22, the output height, uₚ, is a linear function of the input and the output pulse width is t_{pw} that is nominally constant. The transfer function in FIG. 22c can be chosen such that the output corresponding to the energy of the most energetic gamma to be detected is approximately 80% of the maximum output value that can be produced by the pulse shaping circuit. The pulse width can be selected to be about one-half the period corresponding to the bandwidth of the Charge Amplifier.

Pulse Counting Electronics

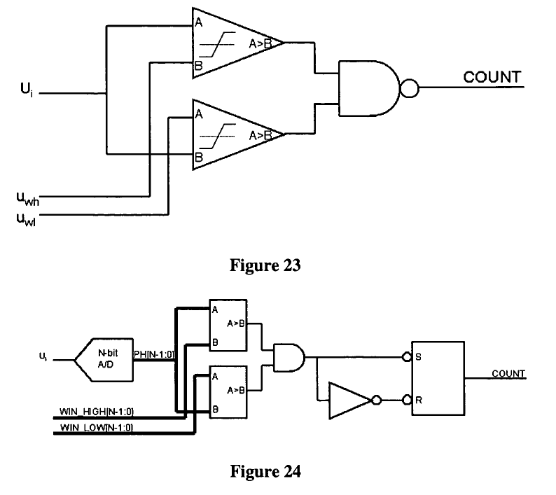

Pulse Counting Electronics can include a plurality of Single Channel Analyzer (SCA) blocks 144. Referring to FIG. 3, the plurality of SCAs can be each connected to the output of the Pulse Shaping Amplifier (PSA). Each PSA can be based on analog or digital comparison schemes. A sample block diagram of a possible analog SCA is shown in FIG. 23 and that of a digital SCA is shown in FIG. 24. In each case, the analog input is u_{I} and a digital pulse COUNT exists. In each case, an upper and lower window limit can be specified and COUNT goes true when the input u_{I} is equal to or between the upper and lower limit.

In the analog system shown in FIG. 23, the precise timing of COUNT and when to advance a software counter is determined in a digital algorithm, which can be resident in the Programmable Control Module. One such algorithm could advance the software counter when COUNT transitions from the False (Low) to True (High) state.

In the digital system shown in FIG. 24, the precise timing of COUNT and when to advance a hardware or software counter can be determined by the SR-latch when COUNT transitions from the False (Low) to True (High) state.

Data Collection and Communication:

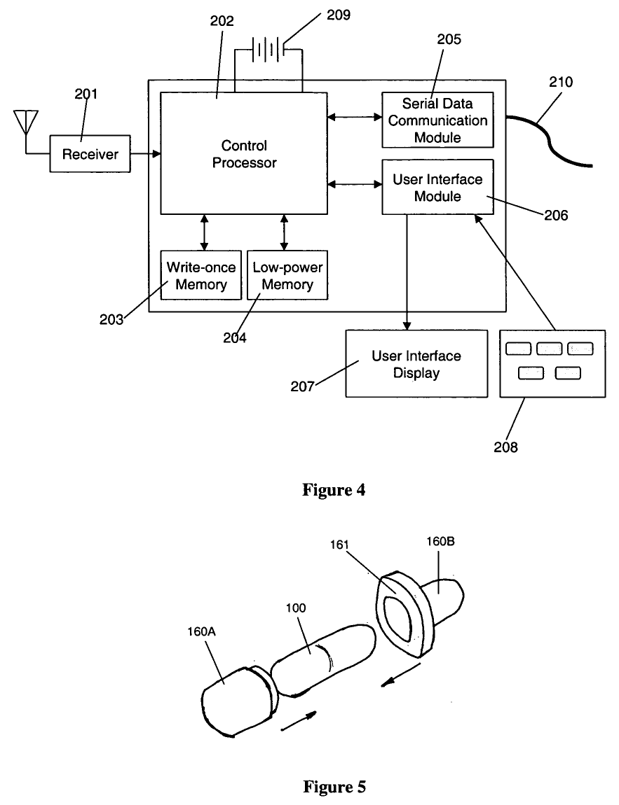

A data collection unit 200 for receiving data transmitted from the transmission module 120 can be employed to store data. The data collection unit can be attached to the patient (such as by clipping on to clothing) or be positioned in a room within receiving distance of the capsule within the patient. Referring to FIG. 4, the data collection unit can include a receiver 201, a control processor 202, a write-once memory 203 for storing configuration information and a unique serial number, a low power memory 204 for storing received data, a serial data communication module 205, a user interface module 206, a user interface display 207, a plurality of control buttons 208, and a battery 209. In one embodiment, the receiver 201, control processor 202, memories 203 and 204, communication module 205, and user interface module 206 can be combined within a single Application Specific Integrated Circuit (ASIC).

The receiver 201 can be selected to be compatible with the transmitter 120 and can convert radio signals to a digital data stream that is applied to the control processor 202.

The control processor 202 can be based on a common commercial microcontroller core such as one based on the Intel 8051 8-bit processor instruction set and architecture. Instructions governing the operation of the data collection unit can be stored in the read-only memory embedded in the control processor core module. The microcontroller core can also provide for the management, control and data transfer between all portions of the ASIC and attached components.

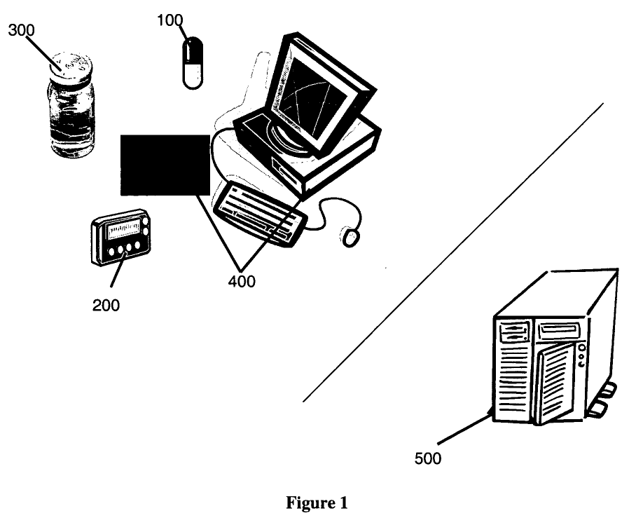

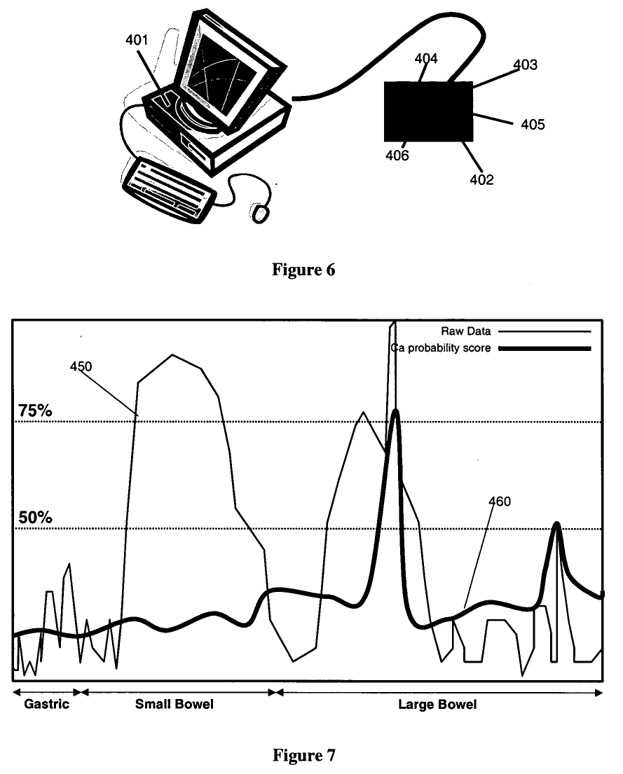

The write-once memory 203 can be used to store configuration information. Configuration information can be entered at the time of manufacturing or through connection to a physician workstation 400 shown in FIGS. 1 and 6. At the time of manufacture various parameters and a unique receiver unit serial number can be stored. When the receiver unit is activated at the physician workstation, other information such as a unique physician identifier code, the capsule serial number, activation date and time, patient number and name, and test type can be transferred to the data collection unit and stored in the write-once memory.

The low-power memory 204 can be used to store data delivered by the capsule. The memory can retain data during any low-power operation modes supported by the control processor and for up to for instance 2 hours when the battery 209 is removed for replacement. Information that can be stored in the memory 204 for each message received from the capsule transmitter 120 can include the time the message arrived, the complete content of the received message and a series of data items to ensure data integrity. Such data integrity information can include data such as a Cyclic Redundancy Check (CRC) word and/or a multi-bit Error Correction Code (ECC).

The serial communication module 205 connects the data collection unit to external computing and communications resources. In one embodiment, the module can contain a serial modem for connection to a telephone subscriber network or to the physician workstation. Alternatively, a USB connection, infrared communications or other standard computer interface can be supplied. To assure compatability with the widest variety of telephone subscriber networks, the data communications rate can be selected to be as low as practicable with 9600 baud signaling considered being sufficient. However, higher data communication rates can also be used.

The user interface module 206 connects to the user interface display 207 and user control buttons 208 to the control processor 202. This module performs any data formatting and device control operations required to efficiently display character and limited graphic information on the user interface display. It also provides appropriate level translation and “de-bouncing” between the user control buttons and the control processor.

The user interface display 207 can be used to present text information and graphics to the user. The display can be of the Liquid Crystal Display (LCD) type with or without backlighting. Various models of the data collection unit can be provided with various levels of graphic and information display sophistication.

The user control buttons 208 can comprise a plurality of “push button” switches. In the preferred embodiment, the switches are all momentary single pole, single throw (SPST) type based on a pressure sensitive membrane switch technology. At least one button can be used to control the power state of the data collection unit.

The battery 209 powering the data collection unit 200 can be relatively inexpensive, such as a 1.5 volt “AAA” battery.

At the conclusion of the testing period (i.e. after the capsule has passed through the patient's entire gastrointestinal tract) the data collected by the data collection unit 200 can be uploaded via an electronic connection, data line or over an internet connection to the central processing center 500 (FIG. 1), or the stored data can be delivered physically by common carrier to a desired location. The data can be transferred to the central processing center 500 directly by the patient (e.g. through an Internet connection or modem connection via a Personal Computer located in the home) or can be transferred by a remote collection and communication facility operated by an agent such as a pharmacy, clinic or physician's office.

Central Processing Center:

The Central Processing Center 500 can be composed of a plurality of substantially identical computing, communication and operator interface resources. The core of the resource pool can be an Internet Server. One or more Internet Server can have a plurality of modems connected to a plurality of telephone subscriber network assets. One or more Internet Server 501 can be dedicated to maintaining the database of capsule and data collection unit serial numbers, physician identification numbers and associated physician information, test performed tests analyzed and billing status. For diagnostic purposes, each Internet Server can be selectively connected to an operator interface unit composed of a plurality of display screens, a keyboard, and pointing device.

When data is communicated to the central processing center 500, it can be processed with a series of data analysis techniques that are used to assess the time sequence of differentiator outputs to identify suspicious data regions. Once analyzed, the capsule serial number is matched with a database of patients, physicians, capsule serial numbers, and procedure type to determine diagnostic report type and electronic address for delivery of electronic reports. If a database match is found, the report is finalized and delivered in a secure, encrypted fashion to the electronic address on record.

Physician Workstation:

Referring to FIG. 6, a physician workstation and analysis system 400 can also be employed. The physician workstation can be based on a standard personal or office computer 401. A capsule interface unit 402 can be provided. For a radiolabeled MAb material 300 (FIG. 1), the interface unit 402 can include a capsule receptacle 403 for receiving the capsule 100 enclosed in protective package 160; a vial receptacle 404 for receiving the marker vial 300 containing the radiolabeled Mab material (shown in FIG. 1); a built-in version of the patient data collection unit 405; and a socket 406 to accept the cable from or directly plug into a Patient Data Collection unit 200. The interface unit 402 can also include an internal communication system such that all components (the capsule 100, vial 300, and data collection unit 200) can be secured in the correct sockets to download the data from the interface unit 402 into the computer 401.

The interface unit 402 can further include one or more barcode readers 410 therein. Barcode reader 410 can be used to read one or more serial numbers on capsule 100, vial 300, and/or data collection unit 200. Barcode readers are well known in the art and one of many suitable barcode readers may be used in interface unit 402.

Computer 401, which can be, but is not limited to, a PC or MAC computer, a workstation computer, or a palm pilot, includes a connection port 412, a user interface 414, and a monitor 416. The connection port 412, which helps connect interface unit 402 to computer 401, can send and receive data to and from capsule 100, vial 300, and/or data collection 200 via interface unit 402. The data sent to computer 401 can be encrypted for security measures. User interface 414 allows a user to enter information into computer 401 and can be, but is not limited to, a standard keyboard or mouse. Computer 401 runs on an operating system, such as, for example, Windows, UNIX, MacOS, Linux, Palm OS, among others. Computer 401 further includes at least one software program loaded on it used to analyze and communicate with the interface unit 402 including capsule 100, vial 300, and data collection 200. The software program, which can be written in computer languages, such as, for example, Java, C++, Visual Basic, among others, can include a Graphical User Interface used to graph the data received from the capsule 100, vial 300, and data collection 200. The software program can further include a decryption code used to decode any encrypted data sent from the interface unit 402.

The interface unit 402 can be connected to the computer 401 via any one of a number of standard computer peripheral methods such as, but not limited to; an RS232 serial interface, an IEEE1394 or USB interface, via an ethernet cable or phone line over the Internet or a Local Area Network, a parallel printer-like data interface, a fiber optic interface, a custom PCI card interface, or an infrared or RF interface. The software program in the computer 401 can also be used to facilitate operation of the interface unit 402.

Functions that can be provided by the workstation 400 include but are not necessarily limited to 1) verify the operability of the capsule 100; 2) verify the operability of the data collection unit 200; 3) verify the activity level of the differentiator (such as a radio-labeled MAb embodiment); 4) program patient, physician and test type information into the data collection unit 200; 5) communicate, via a secure, encrypted data method, with the data collection facility 500 the name and ID of the physician and patient, the serial numbers of the capsule and the data collection unit, type of test requested and administered, and time of injection.

It can be a further function of the physician workstation to receive encrypted secure data report from the data collection and analysis center 500 and subsequently display or print that report on demand.

To acquire the several pieces of data to be entered by the physician or an associate, a modern user interface, such as a graphical user interface, can be provided for operation on the computer 401.

To activate and/or verify operability of the capsule 100, the interface unit 402 socket or port that is adapted to accept the capsule complete with its protective package 160 can include an activation mechanism, such as a magnetic means (assuming that the capsule power is magnetically activated) to override the field created by the magnet contained in the protective package. The built-in data collection unit 405 can receive and/or respond to data provided by or stored in the capsule and provide that data to the control computer 401 for performing basic data validation checking.

To verify operability of the patient's data collection unit 200, the unit 200 can be connected to the workstation interface 402 via the data collection unit interface cable 210 (FIG. 4). With the capsule 100 transmitting data, the output from the patient data collection unit 200 can be compared with the output from the built-in data collection unit 405.

To verify the activity level of the differentiator (radio-labeled MAb) material 300, the vial of material 300 can be inserted into a the mechanical socket provided in the interface unit 402. With the capsule 100 also inserted in its mechanical socket, the radioactive count levels received by the capsule from the vial of material 300 can be transmitted to the built-in data collection unit 405 and the patient data collection unit 200. The information can then be communicated to the computer 401 to be checked against a range of acceptable values.

After verifying correct operation of the various system components (i.e. capsule 100, patient data collection unit 200 and the differentiator), physician entered data and various calibration and configuration codes determined by the software plus patient information can be transmitted to the patient data collection unit via the data collection unit interface cable 210. Within the patient data collection unit 200 this data can be stored in an appropriate location within the write-once memory 203.

FIG. 7 shows a typical report as it might be displayed in written or electronic form at the physician workstation. On this report, the raw data corresponding to radiation counts per unit time received by the detector is normalized and presented as raw data curve 450 with respect to the approximate location in the GI tract indicated on the horizontal axis. As a result of data processing that takes place at the central data collection facility 500, a predictive score can be provided (such as is depicted as curve 460 in FIG. 7). The importance of the predictive score can be determined by clinical reports and the experience of the physician analyzing the results. In general, the purpose of the predictive score can be to indicate if a peak in the raw data curve 450 indicates cancer or background radiation such as from the material 300 stored in the liver or spleen. For instance, in FIG. 7, the peak in the raw data curve 450 corresponding to the small bowel is not likely to indicate the presence of cancer in the small bowel due to the probability value provided by the curve 460 corresponding to the small bowel.

Differentiator Alternatives

To improve the sensitivity of the test results, alternative and additive methods could be adopted.

Two Differentiator Method

In a different embodiment, two or more differentiator agents can be used in order to increase the accuracy of the test. Currently, the accuracy of a differentiator such as a monoclonal antibody is limited by their distribution to healthy organs as well as disease areas. For example, monoclonal antibodies tend to distribute to the liver, kidneys, spleen, urinary bladder and bone marrow. This can give rise to false positive readings, or reduced specificity, since signals emitting from one of those organs are falsely interpreted as emanating from disease. Moreover, the radioactivity coming from the circulating portion of the injected MAb may be much higher than that emanating from a small tumor or lesion, thus masking the real diseased tissue. The physician is then unsure as to the nature of the signal: is it emanating from diseased cells, or does it merely represent normal distribution of the antibody throughout the body?

Rather then only receiving one differentiator, for example a radiolabled MAb specific to disease, the patient also receives a similar MAb, albeit one which is tagged by another particle. For example, if the original drug were a MAb marked with radioactive material such as ^{99m}Tc, then the co-administered agent could be a similar MAb tagged with a different radioactive label, such as ¹¹¹In. Moreover, the second agent could be designed so as to concentrate in similar concentrations in the different body compartments (e.g. kidney, liver, blood, and liver). To this end, the second agent could have similar molecular weight, charge and physical characteristics, but would have a different binding surface. A practical way to achieve this could be to use two monoclonal antibodies of the IgG type, one with specificity to the tumor tagged with ^{99m}Tc, the other being a non-specific IgG antibody tagged with a different radioactive marker such as ¹¹¹In.

Upon administration to the patient, both MAb's will concentrate in equal amounts within the body compartments. However, there will also be some tumor uptake of the MAb that is designed to attach to the tumor. Using a radioactivity analyzer that can different between the isotopes, one can determine for each area of the body how much radioactivity is emanating from each of the two labels. Since the labels are designed or chosen so as to have similar molecular weight and composition, they are per-definition very similar in their pahramacokinetic and pharmacodynamic qualities. Thus, by subtracting the radioactivity intensity emanating from one source from that coming from the other one should get a negligible reading of radioactivity. This will generally be the case, except where there is a tumor to which one of the antibody types attaches, in which case this MAb will have stronger binding and the radioactivity emission from this area will be markedly higher than that coming from the isotope attached to the second antibody. The final response to the physician can be the net result of subtracting the two radioactivity levels, which may significantly reduce confusion associated from background interference, or the non-specific distribution explained above.

The method of this embodiment can include the following steps:

1. A specific differentiator for a tumor or another abnormal tissue such as inflammatory or necrotic tissue. Possible differentiators include but are not limited to a monoclonal antibody, peptide, nucleic acid (nucleotide), nano-particle, or other.

2. A marker material which is bound to the differentiator or that binds to it upon administration to the patient. Possible materials include but are not limited to radioactive nuclides such as ^{99m}Tc, fluorescent molecules such as one of the porphyrin family of chemicals, ultrasonic contrast agents or other.

3. An agent similar to agent (1) in physical and electrical aspects, for example a protein of similar molecular weight, charge and 3-D structure. This agent is different from that in 1 in that it does not attach to the same moiety in the body. To illustrate, if a MAb from the IgG immunoglobulin class is chosen, such as the commercial drug Oncoscint, a good agent to choose as the second agent (3) would be a IgG antibody that is not specific to a known moiety in the body. Alternatively, one can use or a mixture of non-specific IgG. Finally, one can choose an IgG whose Fc portion or antigen recognition area is engineered so as not to fit a specific receptor. For example, an IgG antibody whose Fc portion consists of a repetitive sequence of one amino acid, such as Alanine.

4. A marker material bound to the agent in (3), which is different from that in 2. For example, if the radioactive isotope ^{99m}Tc was chosen above (2) then the isotope ¹¹¹In can be chosen here.

5. A system which detects the signals emitted by markers (2) and (4), be it a radioactivity detector, magnetic field sensor, or other signal. The system should be able to differentiate between the two different sources. For example, radioactivity resulting from the presence of ^{99m}Tc should be differentiated from that resulting from ¹¹¹In due to the widely separated decay energy of the respective gamma radiation.

6. The signals coming from the two markers are subtracted or otherwise processed and the result is exhibited to the user.

This method increases the value of diagnostic tests, by reducing the false negative rate.

As a result of less false positive tests, the system will reduce the unnecessary ensuing tests, thus reducing their associated cost.

The method may also allow the user to increase the level of differentiator given to patient in order to increase its sensitivity, without worrying about increasing noise. Thus, the system can increase both sensitivity (e.g. what proportion of patients are diagnosed) and specificity (given a positive result, what is the likelihood that that patient is indeed sick)

Avidin/Biotin Method

In another embodiment, in order to increase test accuracy one may use materials that strongly bind to each other, but have less binding affinity or none at all to other chemical moieties. Apart from antibodies mentioned above, other materials that have relatively high binding affinity to each other can be used. In nature, or when mixed together under laboratory conditions, such agents will strongly bind to each other in a tight, nearly permanent fashion.

The most commonly known of these couples is the Avidin-Biotin couple. Biotin is a vitamin from the B complex. It is a colorless crystalline vitamin with chemical composition C₁₀—H₁₆—N₂—O₃—S. It is essential for the activity of many enzyme systems. Avidin is a protein found in uncooked egg white that binds to and inactivates biotin. This attraction is so firm that an abundance of Avidin in the diet can result in a deficiency of biotin.

Biotin's and Avidin's attraction to each other is often used in laboratory experiments, often for diagnostics. The relationship between Avidin and Biotin has also been used by the pharmaceutical industry in order to develop guiding mechanisms for drugs [See Karacay H, et al. Development of a streptavidin-anti-carcinoembryonic antigen antibody, radiolabeled biotin pretargeting method for radioimmunotherapy of colorectal cancer. Reagent development. Bioconjug Chem 1997 Jul-Aug;8(4):585-94, and Schultz A. Tetravalent single-chain antibody-streptavidin fusion protein for pretargeted lymphoma therapy. Cancer Res 2000 Dec. 1;60(23):6663-9 which are incorporated herein by reference].

Other proteins with similar structure as Avidin or derivatives thereof may be used in order to optimize its binding, reduce clearance, improve its pharmacokinetic or pharmacodynamic attributes or induce other favorable effects. For example, Recombinant Streptavidin (rSAv) may be used instead of Avidin. Furthermore, it may be necessary to modify rSAv in order to get a more favorable action, for example by reducing its rather high kidney localization. Methods that have been described in the medical literature to that end include succynilation of rSAv using Succinic Anhydride [Wilbur DS, et al. rSAv in antibody pretargeting. 3. Comparison of biotin binding and tissue localization of 1,2-cyclohexanedione and succinic anhydride modified recombinant streptavidin. Bioconjug Chem 2002 May-Jun;13(3):611-20], administration of I-Lysine [Wilbur DA, et al. Streptavidin in antibody pretargeting. 2. Evaluation Of methods for decreasing localization of streptavidin to kidney while retaining its tumor binding capacity. Bioconjug Chem 1998 May-Jun;9(3):322-30], which are incorporated herein by reference.

In one embodiment, a method can be used to employ the association between Biotin and Avidin or other similar “couples” in order to increase the accuracy of capsule-based cancer diagnosis. The method can include the following steps:

1. The patient first receives a MAb or FAb or another differentiating molecule specific to disease such as cancer. Attached to the MAb is Avidin or Streptavidin, or another member of the Avidin family. Attachment of the Avidin or Avidin-like moiety to the MAb or FAb or other agent used as the differentiator may be achieved by genetic engineering creating a fusion protein [as described by Schultz A. Tetravalent single-chain antibody-streptavidin fusion protein for pretargeted lymphoma therapy. Cancer Res 2000 Dec. 1;60(23):6663-9, incorporated herein by reference].

2. After allowing the drug to accumulate in diseased tissue, the patient is then given a clearing agent containing biotin or another molecule with very high affinity to the initial agent. Biotin binds strongly to the drug given in step 1 and is still free in the body. Thus, any remaining drug is that which is bound to the specific target. Alternatively, in another embodiment one may wait ample time for the drug given in step 1 to naturally clear from the body.

3. The patient receives a biotin attached to a radioactive or other marker such as ^{99m}Tc, a magnetic particle, a fluorescent marker, or other marker. The Biotin binds the Avidin and marks the disease with radioactivity or another mode, depending on the marking agent attached to Biotin.

4. The patient is given the capsule before, during or after the above procedure. The capsule contains the sensing device, for example, a radioactive detector. This method increases the value of diagnostic tests, by reducing the false negative rate.

Operation

The following operational description refers to devices and methods of the present invention wherein a cell marker material comprising a radio-labeled monoclonal antibody is employed. For purposes of screening a target population for colon cancer in a relatively non-invasive procedure, the following operational steps can be employed.

A patient requiring screening can present to a physician or physician associate for a colorectal cancer screening test. Prior to arrival, the physician or related staff can order and receive a screening kit from a pharmacy licensed to dispense nuclear medicine materials and taken delivery of that test kit earlier on the date of the patient visit.

Upon arrival of the patient, the physician can place components of the kit in a special fixture at the workstation 400. The components of the kit can include a swallowable detection capsule 100, a patient data collection unit (PDU) 200, and an injectable cell marker material (CM) 300. The physician workstation and associated software can be used to verify the operability of all of the kit components and programs certain information into the PDU.

Once the kit is determined to be operable, the physician can inject the cell marker material 300 into the patient and the patient can be instructed to swallow the detection capsule. The patient can be instructed on the use of the PDU and it can be attached to the patient in the same fashion as a pager, cell phone or wrist watch.

At this point, the patient returns to normal daily activity as the capsule and detetctor travel through the GI tract from the esophagus through the stomach, small intestine, colon (large intestine) and eventually is expelled through the anus with stool during a bowel movement.

As the detector travels through GI tract, it is periodically measuring and reporting radiation emitted from various sources in the patient. This information can be combined with a unique identifier code for the Detector and a timing indication as it is transferred to the PDU. The PDU can be used to collect and store all of the information from the detector for subsequent communication to the data collection and analysis center (DCAC) 500.

Once the data arrives at the DCAC, a series of analytical routines can be applied to the raw data and a procedure specific report can be generated. That report can be routed to the physician (such as to the physician workstation) and can include information that verifies operability of the kit and encodes the patient and physician information into the PDU.

It will be recognized that equivalent structures may be substituted for the structures illustrated and described herein and that the described embodiment of the invention is not the only structure which may be employed to implement the claimed invention. In addition, it should be understood that every structure described above has a function and such structure can be referred to as a means for performing that function.

While preferred embodiments of the present invention have been shown and described herein, it will be obvious to those skilled in the art that such embodiments are provided by way of example only. Numerous variations, changes, and substitutions will now occur to those skilled in the art without departing from the invention. Accordingly, it is intended that the invention be limited only by the spirit and scope of the appended claims.

What is claimed is:

1. A method for detecting target cells in a patient comprising:
a) marking target cells in the body with a signal emitting substance
b) directing a detector through a naturally occurring body lumen in the patient to detect the signals; and
c) differentiating between signals associated with target cells and signals associated with non target cells.

2. A method for detecting target cells in a patient comprising:
a) administering to a patient a material comprising at least one signal emitting substance and at least one substance having an affinity for a target cell type.
b) providing a detector capable of detecting signals emitted by the substance;
c) directing the detector through the patient's gastrointestional tract;
d) differentiating between signals associated with the target cells and signals associated with non target cells.

3. A method comprising the steps of:
a) administering to a patient a material capable of targeting a target cell type;
b) administering to the patient a clearing agent for removing the material which is not bound to the target cell type;
c) directing a detector through the patient's gastrointestinal tract to detect the target cell type.
